(19) United States
(12) Patent Application Publication
(10) Pub. No.: US 2020/0229757 A1
(43) Pub. Date: Jul. 23, 2020

(54) METHOD AND SYSTEM FOR MONITORING THE PROGRESS OF TREATMENT OF AN INDIVIDUAL HAVING ACUTE PAIN, CHRONIC PAIN, ACUTE STRESS DISORDER, BLAST EXPOSURE OR PTSD USING SPECTRAL DATA OF THE BRAIN

(71) Applicant: Translational Research Institute Pty Ltd as trustee for Translational Research Institute Trust, Woolloongabba (AU)

(72) Inventor: Carolyn Mountford, Robina (AU)

(73) Assignee: Translational Research Institute Pty Ltd as trustee for Translational Research Institute Trust, Woolloongabba (AU)

(21) Appl. No.: 16/661,689

(22) Filed: Mar. 26, 2020

Related U.S. Application Data
(60) Provisional application No. 62/750,014, filed on Oct. 24, 2018.

(51) Int. Cl. A61B 5/00; A61B 5/145; A61B 5/1455; A61B 5/16

(52) U.S. Cl. CPC A61B 5/4824; A61B 5/14532; A61B 5/1455; A61B 5/4064; A61B 5/0022; A61B 5/4848; A61B 5/165; A61B 5/7246; A61B 5/4836

(57) ABSTRACT

A method and system obtains spectral data from the brain of a person undergoing treatment for pain to determine whether the concentration of new chemicals including fucosylated glycans indicates fucosylated glycan concentration levels of a person experiencing pain or returning to normal levels of a healthy person who is not experiencing pain, to assess the person's response to treatment and progress.

### CROSS-REFERENCE TO RELATED APPLICATIONS

This application claims priority to U.S. Ser. No. 62/750,014 filed Oct. 24, 2018, which is incorporated by reference herein.

### BACKGROUND OF THE INVENTION

The present invention relates to a method and system for monitoring the progress of treatment of an individual who has detectable abnormal activity in the brain indicative of acute pain, chronic pain, acute stress disorder, blast exposure or PTSD (post traumatic stress disorder).

References are cited herein as background to aid in understanding the invention. These references are incorporated by reference herein.

Individuals who suffer from various conditions and diseases seek treatment or therapy in order to improve their condition. Typically, the condition is reported on a subjective scale from 1 to 10 for example, but is not objectively measured. After treatment or therapy, which may include the mere passage of time, an individual may report their condition on a subjective scale again, but there is no objective way of determining whether the condition is improving as a consequence of time or therapy. The subjective reporting may be inaccurate, and therefore it is difficult to determine whether the treatment is actually working, and if so the rate of recovery which information should be employed to aid in the recovery.

Temporomandibular disorders (TMD) involve the masticatory muscles, temporomandibular joints and related structures (1). The aetiology of TMD is complex and the Diagnostic Criteria for Temporomandibular Disorders (DC/TMD) has enabled taxonomic classifications. These include temporomandibular joint disorders and sub-classifications; masticatory muscle disorders and sub-classifications; headache and other associated structures (2). These disorders can cause pain and discomfort, functional changes such as joint noises due to wear and tear, and structural changes including atypical jaw movements. Approximately 90% of the general population will be affected by TMD at some stage of their life with a higher prevalence in females aged 20-40 (3).

Low back pain (LBP) carries the greatest burden of disease in Australia and is associated with the highest years lived with disability globally (12). It is the third most common health complaint seen by general practitioners, and the total cost annually of LBP in Australia is estimated to be A$1 billion (13). Most acute LBP is non-specific, caused by muscle or ligament injury with the majority of patients improving within the first month (14).

Two dimensional (2D) COrrelated SpectroscopY (COSY) of the posterior cingulate gyrus and other regions of the brain has demonstrated its ability to determine different types of pain by analysing degrees of neurochemical deregulation (4, 5).

Five fucose-α(1-2)-galactose sugars (glycans) have recently been assigned in the human brain (6) and are expressed as terminal saccharide glycoproteins and glycolipids (7). These fucosylated glycans are affected by the pain process. They have been shown, by others in animal models, to be implicated in the mechanisms underlying neuronal development, learning, memory (8); regulation of nervous system development and function (9); and to influence various neuronal processes including neurite outgrowth and morphology (8, 10). They are important new molecules to monitor in the evaluation of pain.

### SUMMARY OF THE INVENTION

In accordance with the present invention, a method and system are provided for objectively monitoring the progress of recovery of a person having an abnormal brain condition, such as acute pain, chronic pain, acute stress disorder, blast exposure or PTSD.

The concentration of fucosylated glycans, and other chemicals, in the individual's brain can be monitored to determine the response of the individual to treatment, including mere passage of time, so that an objective measure can be obtained on whether the individual is undergoing recovery and if so the rate of recovery.

The concentration of fucosylated glycans in the brain change from normal levels indicative of a healthy state, to abnormal levels when the individual experiences acute pain, chronic pain, acute stress disorder, blast exposure and PTSD. By using 1D or 2D COSY magnetic resonance, the concentration levels of the fucosylated glycans can be detected to determine whether the individual is undergoing recovery and if so the rate of recovery. The fucosylated glycans can be identified by spectral analysis of data obtained in a magnetic resonance scanner.

As the individual recovers from their condition, it has been determined that their fucosylated glycan levels, which were not normal in their disease or condition state, return to their normal state. Some glycans which were depleted undergo repopulation during recovery, and some glycans which increased are returned lower to their normal levels. Accordingly, an objective measure can be obtained on the rate of recovery, and when an individual is fully recovered, which is much more accurate that a subjective self-reporting by an individual used previously. By monitoring progress objectively an individual knows which therapy or treatment, including mere passage of time, is effective for him or her, with a degree of certainty missing from the subjective self-reporting used previously.

As used herein, the terms “acute stress disorder” refers to a condition acute stress where an individual experiences symptoms such as, without limitation, feeling nervous, restless or tense; has difficulty controlling worry; feels weak or tired and/or has trouble sleeping.

### BRIEF DESCRIPTION OF THE DRAWINGS

FIG. 1 is a block diagram of a system which can be used to obtain the magnetic resonance spectroscopy data;

FIG. 2A shows a 3D map of a fucose region of spectra obtained from a healthy control subject;

FIG. 2B shows a 2D contour map of the fucose region of the spectra of FIG. 2A;

FIG. 2C shows a 3D map of a fucose region of spectra obtained from a subject having chronic TMJ (temporomandibular joint);

FIG. 2D shows a 2D contour map of the fucose region of the spectra of FIG. 2C;

FIG. 2E shows a 3D map of a fucose region of spectra obtained the same subject as in FIGS. 2C and 2D with chronic TMJ 6 days post treatment;

FIG. 2F shows a 2D contour map of the fucose region of the spectra of FIG. 2E;

FIG. 3 shows the peak volumes of the fucose region for pre-treatment and post-treatment for TMJ, with blue (circle) indicating pre-treatment and orange (square) indicating post-treatment;

FIG. 4 shows placement of a 2D voxel in posterior cingulate gyms (PCG) on an MR image for a patient experiencing lower back pain (LBP);

FIG. 5A shows a 3D surface plot from the spectral region 4.0-4.6 (referred to as the fucose region) for a subject with acute LBP 30 hours post injury;

FIG. 5B shows a 2D contour plot of the same fucose region as in FIG. 5A, for 0.9-1.6 ppm;

FIG. 5C shows a plot similar to FIG. 5A, but for the subject 4 weeks post injury;

FIG. 5D shows a plot similar to FIG. 5B, but for the subject 4 weeks post injury;

FIG. 5E shows a plot similar to FIG. 5A, but for the subject 8 weeks post surgery;

FIG. 5F shows a plot similar to FIG. 5B, but for the subject 8 weeks post surgery;

FIG. 6 shows the peak volumes of the fucose region for the LBP study, with blue (circle) indicating 30 hours post injury onset, orange (square) indicating 4 weeks post injury and green (diamond) indicating 8 weeks post injury onset.

### DETAILED DESCRIPTION OF A PREFERRED EMBODIMENT

A preferred embodiment of the invention will be described for both TMJ and LBP types of pain, but the invention is not limited to this embodiment.

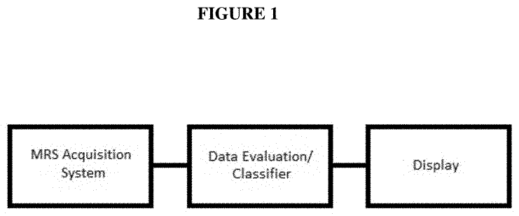

FIG. 1 shows a block diagram of a system which can be used to obtain magnetic resonance spectroscopy data of a person, and can be used to obtain spectroscopy data of a healthy normal person to provide a reference set of spectroscopy data, a person known to be suffering from acute pain, chronic pain, acute stress disorder, blast exposure or PTSD to obtain reference spectral data characteristic of these various stats or conditions, and also to obtain magnetic resonance spectroscopy data of a person whose condition is not known, to determine their condition. The system can also be used to obtain spectroscopy data of a person who has been diagnosed with one of the aforementioned conditions, to determine their response to therapy (which may include the mere passage of time) to determine whether any recovery has occurred returning the person toward a normal healthy state, to aid determining what therapy is having a good effect as well as the progress of therapy.

The results of the spectral data, as will be described, can thus determine, in an objective way, the condition of the person and the rate of recovery, which is usually more accurate than a subjective self-reporting by a person.

### TMJ Case Presentation

A 37-year-old woman with a six year history of chronic TMJ was recruited for this study.

Magnetic resonance spectroscopy was performed at two different intervals using a 3T PRISMA scanner (Siemens Healthcare GmbH, Erlangen, Germany) equipped with a 64-channel head/neck coil. 2D COSY was recorded in the posterior cingulate gyms (PCG) using: RF carrier frequency at 2.0 ppm, TR/TE 1500/30 ms; WET water suppression; spectral width 2000 Hz; increment size 0.8 ms in 96 t1 increments resulting in an indirect spectral width 1250 Hz; 8 averages per increment; 1024 data points and a voxel size of 4×2.5×3 cm.

The participant was first scanned 2 days before she was due to have treatment. Self-reported pain level was eight on a scale of 0-10 (8/10). She was then re-scanned 6 days after treatment. The participant reported that post treatment her pain levels usually took a few days to subside. At the time of the second scan the participant reported that her pain level was zero (0/10).

In the acute phase, visual inspection of the 2D COSY spectrum demonstrated an increase in fucose IV and lactate and a decrease in fucose VI. An overall decrease in the fucose region was seen 4 weeks after initial scan. At the 8 week scan total fucose levels appeared normal. These findings support previous work by Mountford et al on biochemical changes in the brain associated with chronic (lower back pain) LBP (4).

While the patient's self-reported pain levels were nil (0/10) at the 4 week scan the spectrum of the fucose region indicates that the neuro metabolites were still slightly deregulated, that is, at abnormal levels. Mountford et al have previously identified statistically significant differences in the fucosylated glycans in a number of disease cohorts including repetitive head trauma (11), post-traumatic stress disorder and irritable bowel syndrome (unpublished data). This case illustrates that 2D COSY can detect changes to neuro chemistry that occur as deregulation of neuro metabolites from their normal healthy levels takes place during the acute pain phase, but settling back to their normal healthy levels with resolution of symptoms.

### Treatment

The participant was given a therapeutic dose of botulinum toxin A in left and right masseters between the two spectral data acquisitions.

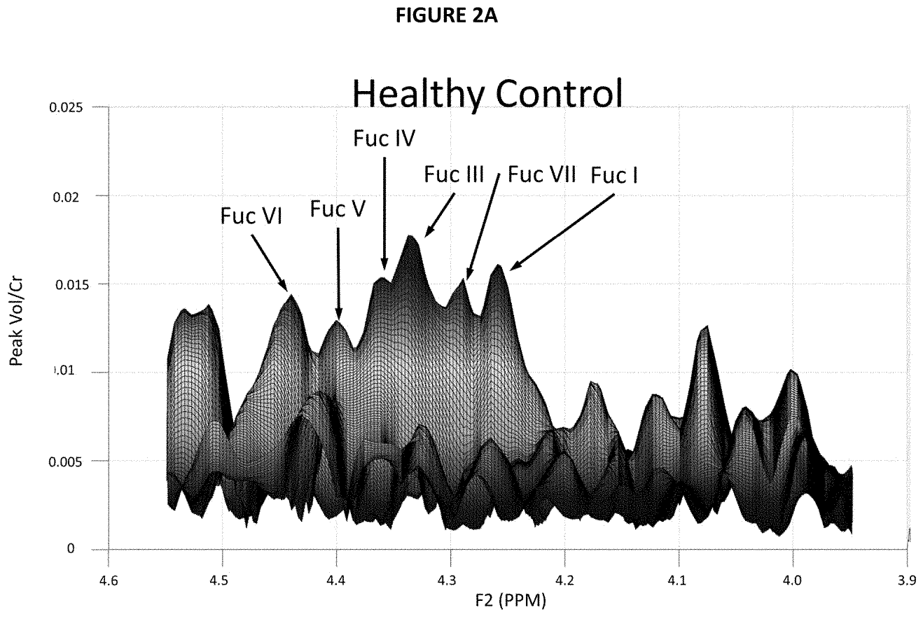

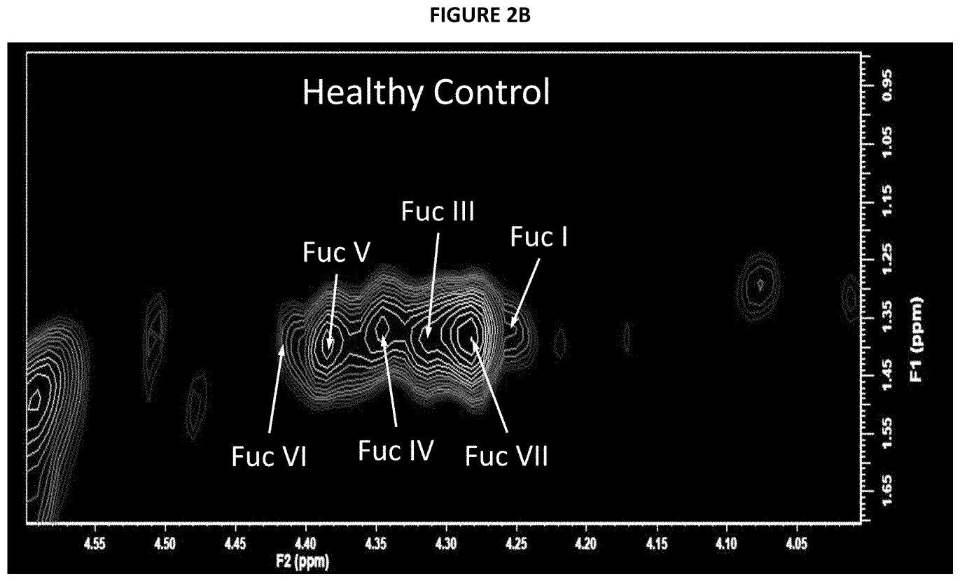

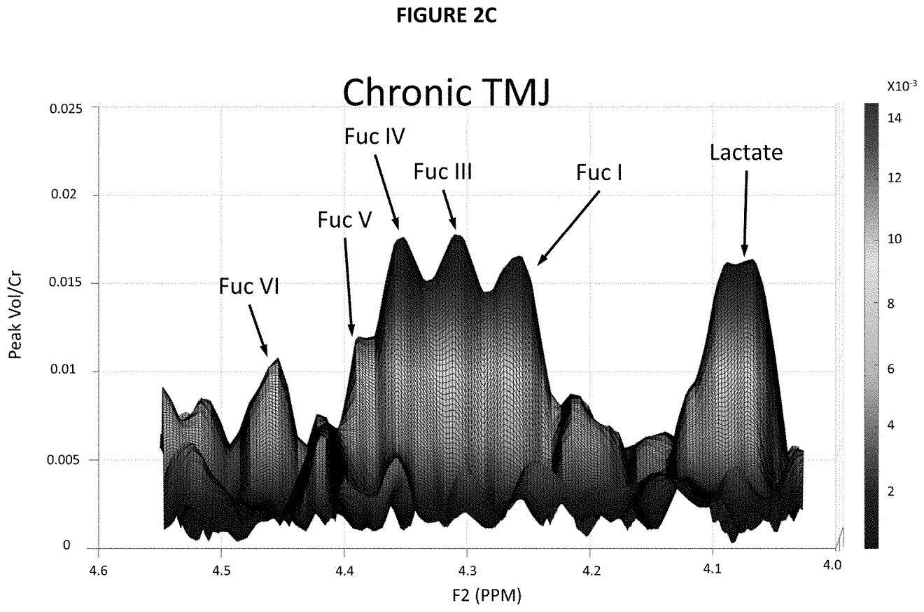

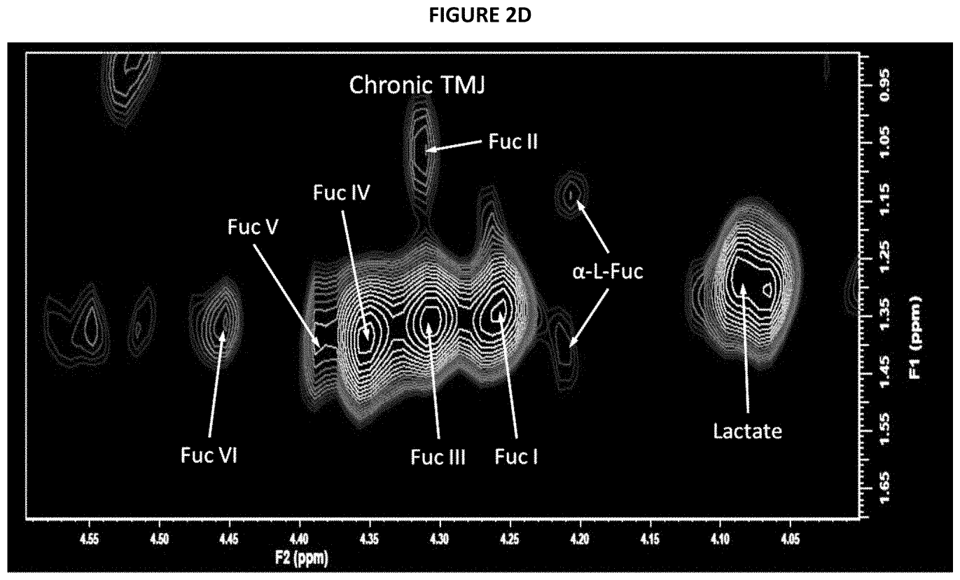

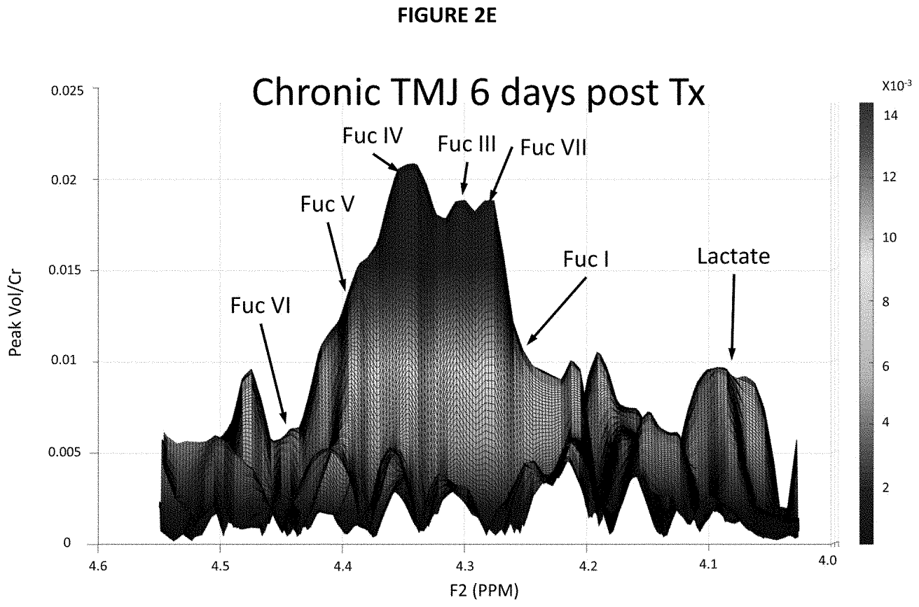

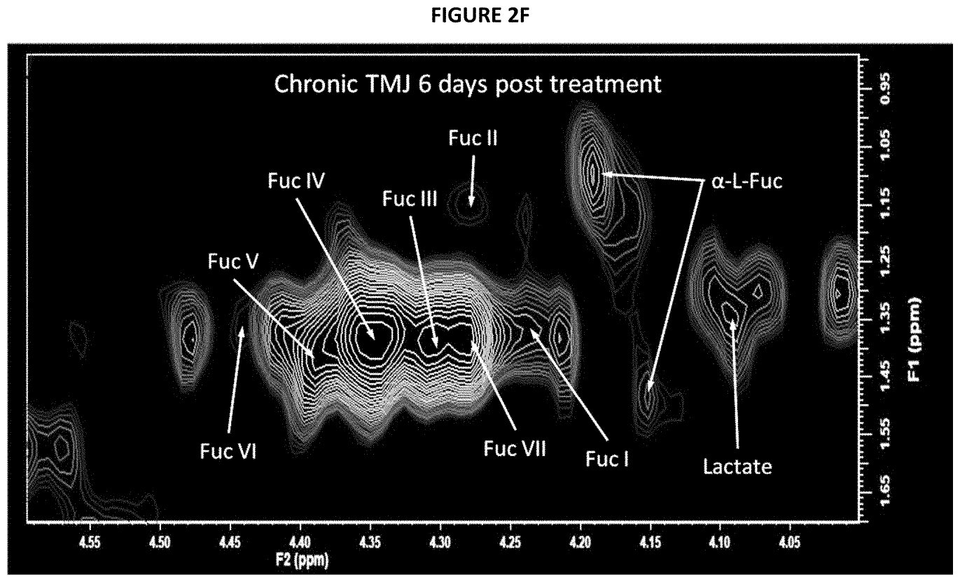

FIGS. 2A-2F show the fucosylated glycans from a spectral data acquisition of the brain before and 6 days after treatment for TMJ. This case study is the first recording a response to therapy.

High levels of free fucose substrate was seen in this subject. This may be the brain attempting to repopulate the affected glycans.

FIGS. 2A-2F show the results of a magnetic resonance spectroscopy spectral data collection of a person in a healthy control state (which provides reference data), a person having chronic TMJ, and the same person 6 days post treatment.

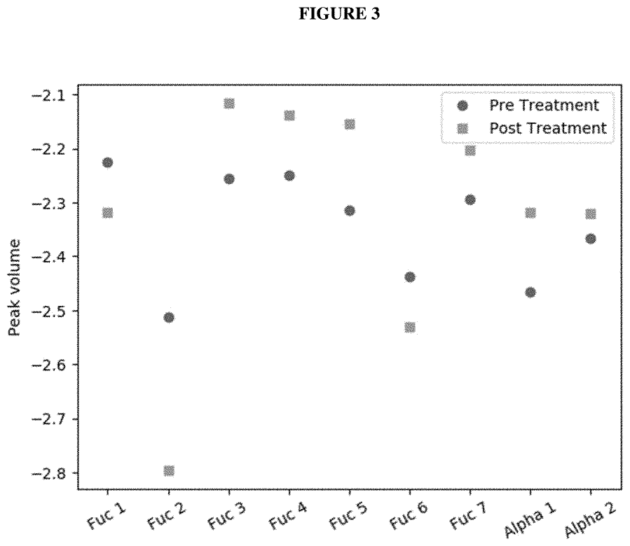

FIG. 3 shows the concentrations of fucosylated glycans and lactate from each of the two data collections. The fucosylated glycans are identified as Fuc I, Fuc II, Fuc III, Fuc IV, Fuc V, Fuc VI, Fuc VII and α-L Fuc. As can be seen, the concentration of Fuc II is relatively low in healthy controls, and rises when the individual is suffering from chronic TMJ. Generally speaking, the concentrations of the other Fuc molecules to include but not limited to (Fuc I, Fuc III, Fuc IV, Fuc V, Fuc VI Fuc VII and α-L Fuc) and lactate decreases when the individual is suffering from chronic TMJ compared to a healthy control, and repopulates back up to normal levels in response to therapy, indicating that the treatment was successful and that the individual has recovered from chronic TMJ.

### LBP Case Presentation

A 33-year-old woman with no history of LBP was recruited for this study. She had a history of tight hamstrings and associated small tears from running. HerLBP began after riding her bicycle to work. She noted central LBP on dismounting with no associated sciatic pain. Stretching or pain relief (ibuprofen) did not alleviate the pain, consequently she sought treatment from a physiotherapist.

The participant was screened by a clinical psychologist to assess any pre-existing conditions that would exclude her from the study. T1 and T2 imaging was performed to rule out any structural brain abnormalities. T2 Magnetic resonance spectroscopy was performed 30 hours after initial onset of pain and then at 4 and 8 weeks post injury using a 3T PRISMA scanner (Siemens Healthcare GmbH, Erlangen, Germany) equipped with a 64-channel head/neck coil. 2D COSY was recorded in the posterior cingulate gyms (PCG) (FIG. 1) using: RF carrier frequency at 2.0 ppm, TR/TE 1500/30 ms; WET water suppression; spectral width 2000 Hz; increment size 0.8 ms in 96 t1 increments resulting in an indirect spectral width 1250 Hz; 8 averages per increment; 1024 data points and a voxel size of 4 cm×2.5 cm×3 cm. At the time of scan 1 the patients' self-reported pain level was 4/10. Pain was nil for the corresponding 2 scans.

### Treatment

The physiotherapy treatment for the LBP plan included exercise, heat and stretching. A 2 week follow up appointment was conducted with a final appointment 10 days later.

### Diagnosis

Impression was facet irritation and mechanical LBP.

### Outcome

The patient's LBP resolved in response to the treatment, as evidenced by the spectral data obtained.

### Discussion

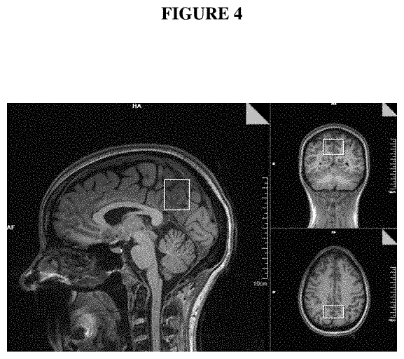

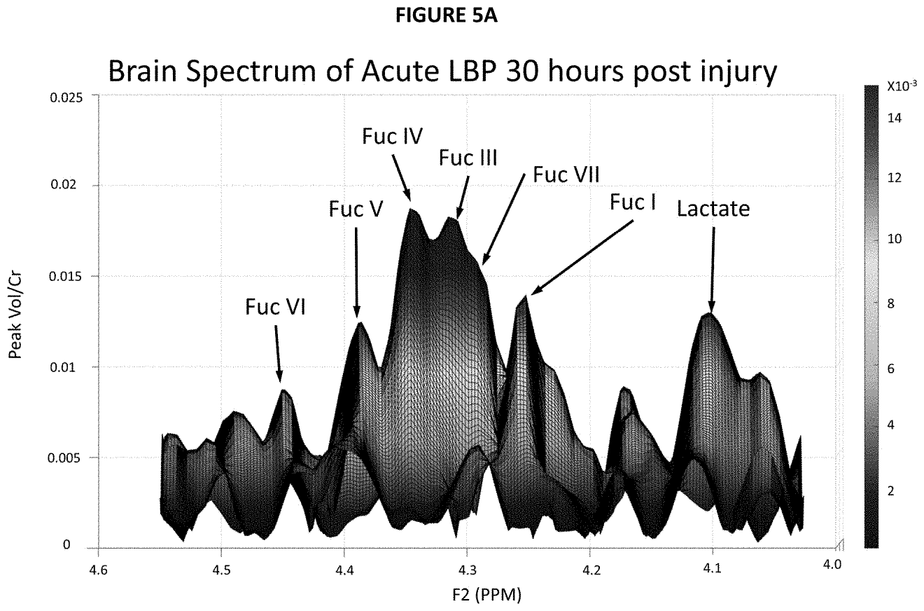

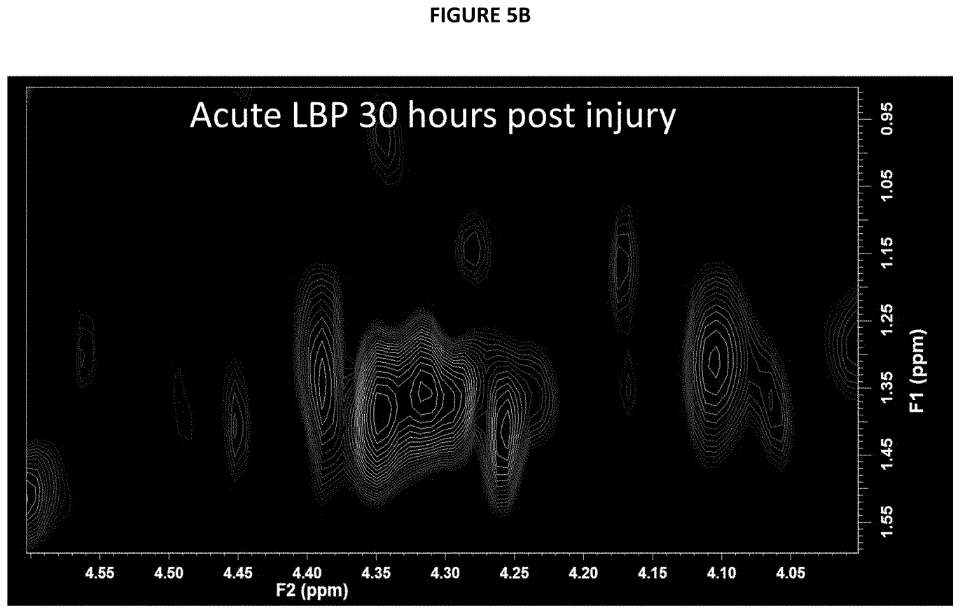

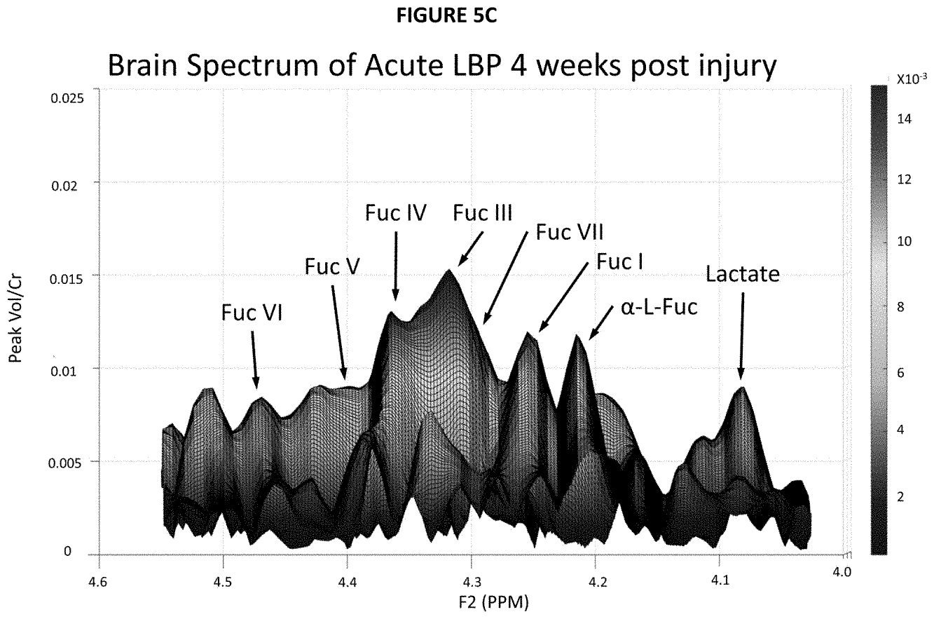

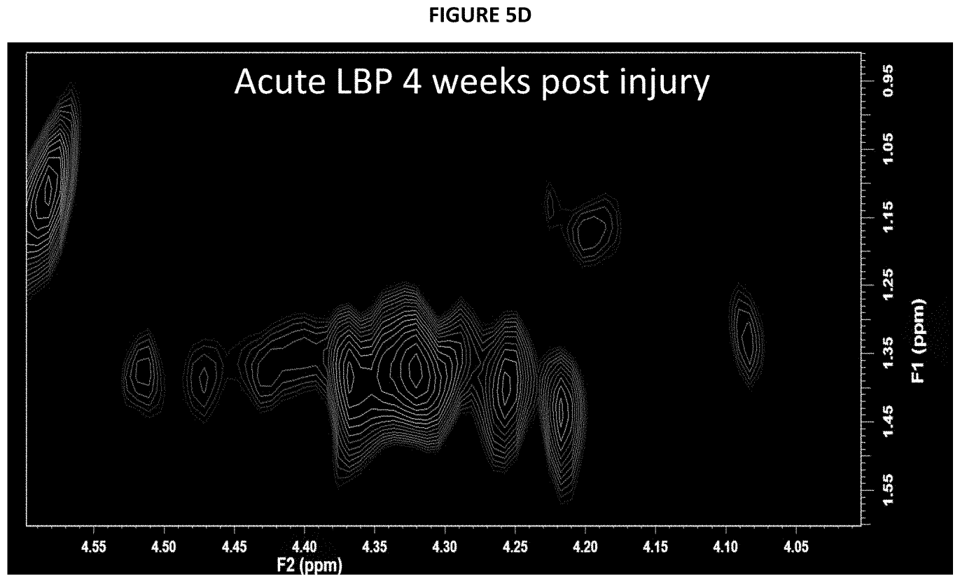

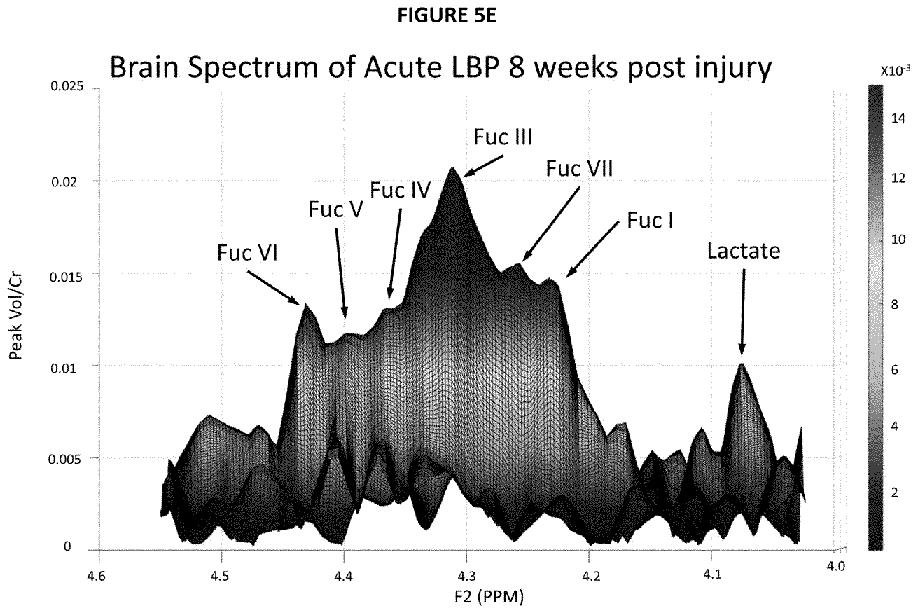

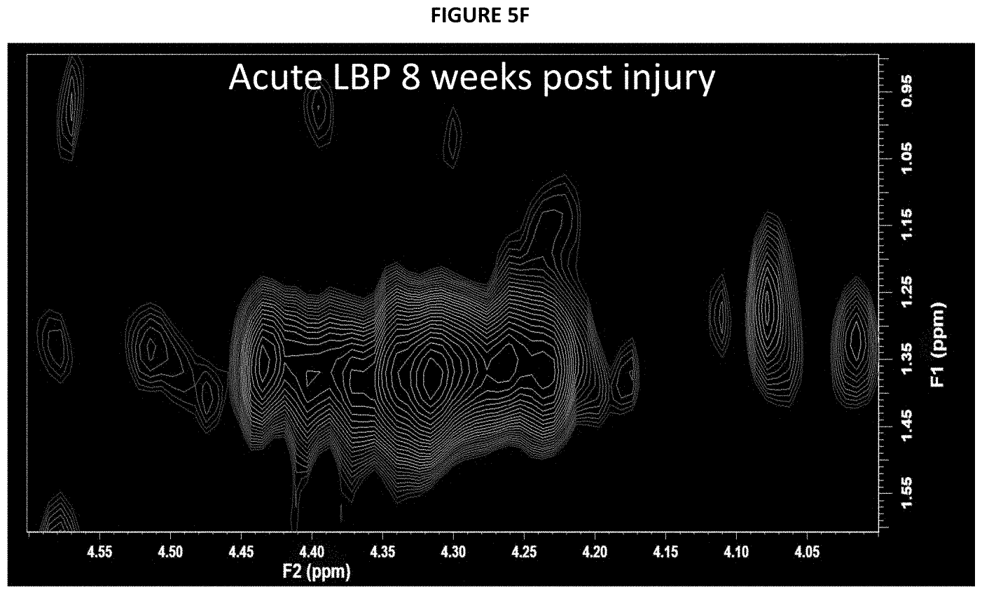

FIG. 4 shows by a box the placement of the 2D voxel in PCG on the MR image. As shown in FIGS. 5A-5F, in the acute phase, visual inspection of the 2D COSY spectrum demonstrates an increase in fucose IV and lactate and a decrease in fucose VI. An upregulation of free fucose substrate is seen in the 2nd scan 4 weeks post injury, with an overall decrease in the remaining fucose region. At the 8 week scan total fucose levels are closer to what we would expect to see in the brain of a healthy person without LBP. These findings support previous work by Mountford et al on biochemical changes in the brain associated with chronic LBP (4).

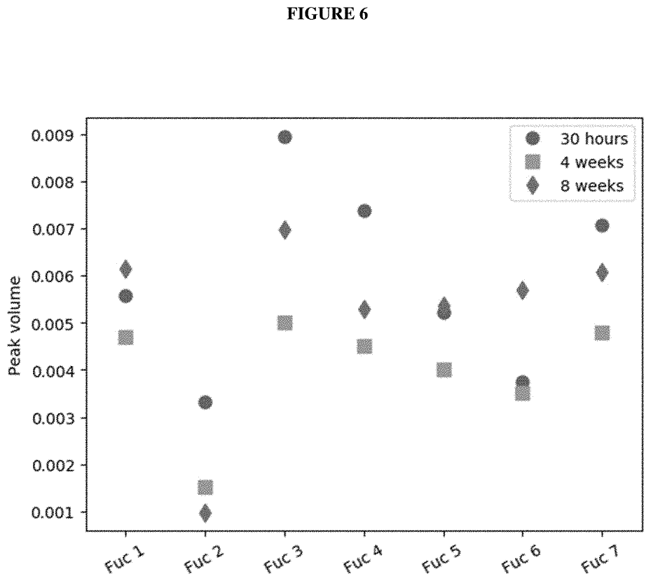

While the patients self-reported pain levels were nil at the 4 week scan the spectrum of the fucose region indicates that the neuro metabolites were still deregulated from their normal healthy levels. Mountford et al have previously identified statistically significant differences in the fucosylated glycans in a number of disease cohorts including repetitive head trauma (11), post-traumatic stress disorder and irritable bowel syndrome (unpublished data). This case demonstrates that 2D COSY to detect LBP by detecting changes in neuro chemistry that occur as deregulation takes place during the acute pain phase, but settling back to normal levels with resolution of symptoms. The results are plotted in FIG. 6.

The same results are expected for acute pain, acute stress disorder, blast exposure and PTSD.

The analysis of the data can occur at a location remote from the location where the data is obtained, and may be done in the cloud after the obtained data is transmitted to the cloud.

A memory device can store program data in non-volatile form for performing program steps to analyse the data.

Although a preferred embodiment has been disclosed, the invention is not limited to this embodiment, and the invention is defined only by way of the following claims.

### REFERENCES

1. Rinchuse D J, Greene C S. Scoping review of systematic review abstracts about temporomandibular disorders: Comparison of search years 2004 and 2017. Am J Orthod Dentofacial Orthop. 2018; 154(1):35-46 e9.
2. Schiffman E, Ohrbach R, Truelove E, Look J, Anderson G, Goulet J-P, et al. Diagnostic Criteria for Temporomandibular Disorders (DC/TMD) for Clinical and Research Applications: Recommendations of the International RDC/TMD Consortium Network( ) and Orofacial Pain Special Interest Group( ) Journal of oral & facial pain and headache. 2014; 28(1):6-27.
3. Laplanche O, Ehrmann E, Pedeutour P, Duminil G. TMD clinical diagnostic classification (Temporo Mandibular Disorders). Journal of Dentofacial Anomalies and Orthodontics. 2012; 15(2):202.
4. Siddall P J, Stanwell P, Woodhouse A, Somorjai R L, Dolenko B, Nikulin A, et al. Magnetic resonance spectroscopy detects biochemical changes in the brain associated with chronic low back pain: a preliminary report. Anesthesia and analgesia. 2006; 102(4):1164-8.
5. Stanwell P, Siddall P, Keshava N, Cocuzzo D, Ramadan S, Lin A, et al. Neuro magnetic resonance spectroscopy using wavelet decomposition and statistical testing identifies biochemical changes in people with spinal cord injury and pain. Neuroimage. 2010; 53. (2):544-52.
6. Mountford C, Quadrelli S, Lin A, Ramadan S. Six fucose-all-2) sugars and α-fucose assigned in the human brain using in vivo two-dimensional MRS. NMR in biomedicine. 2015; 28(3):291-6.
7. Becker D J, Lowe J B. Fucose: biosynthesis and biological function in mammals. Glycobiology. 2003; 13(7):41R-53R.
8. Murrey H E, Ficarro S B, Krishnamurthy C, Domino S E, Peters E C, Hsieh-Wilson L C. Identification of the plasticity-relevant fucose-alpha(1-2)-galactose proteome from the mouse olfactory bulb. Biochemistry. 2009; 48(30):7261.
9. Murrey H E, Hsieh-Wilson L C. The chemical neurobiology of carbohydrates. Chemical reviews. 2008; 108(5):1708.
10. Ralf K, Melitta S. Glycans and neural cell interactions. Nature Reviews Neuroscience. 2004; 5(3):195.
11. Lin A P, Ramadan S, Stern R A, Box H C, Nowinski C J, Ross B D, et al. Changes in the neurochemistry of athletes with repetitive brain trauma: preliminary results using localized correlated spectroscopy. Alzheimer's Research & Therapy. 2015; 7:13.
12. Hoy D, March L, Brooks P, Blyth F, Woolf A, Bain C, et al. The global burden of low back pain: estimates from the Global Burden of Disease 2010 study. Ann Rheum Dis. 2014; 73(6):968-74.
13. Goergen S M C, Leech M, Kuang R. Acute Low Back Pain. Education Modules for Appropriate Imaging Referrals. Royal Australian and New Zealand College of Radiologists; 2015.
14. Lateef H, Patel D. What is the role of imaging in acute low back pain? Current Reviews in Musculoskeletal Medicine. 2009; 2(2):69-73.

1. A method for monitoring the response to treatment of an individual experiencing pain by detecting the level of chemical activity in the brain; comprising
a. obtaining first spectral data from the individual's brain to determine the concentration of fucosylated glycans in the brain;
b. subjecting the individual to therapy, which may include mere passage of time;
c. after step b., obtaining second spectral data from the individuals' brain to determine the concentration of fucosylated glycans in the brain; and
d. comparing the second spectral data to the first spectral data to determine whether the concentration of fucosylated glycans in the second spectral data are changed from the concentration in the first spectral data and returning to the concentration of fucosylated glycans in the brain of a healthy person who is not experiencing pain.

2. The method of claim 1, wherein the pain comprises at least one of acute pain, chronic pain, acute stress disorder, blast exposure and PTSD.

3. The method of claim 1, wherein the first and second spectral data are obtained using 2D COSY.

4. The method of claim 1, wherein the first and second spectral data determine the concentration of fucosylated glycans in a fully free form.

5. The method of claim 1, wherein the first and second spectral data determine the concentration of fucosylated glycans attached to another small molecule as a base pair.

6. The method of claim 1, wherein the step of comparing is performed at a location remote from where the spectral data is obtained.

7. The method of claim 6, wherein the remote location is in the cloud.

8. A non-volatile memory for storing program steps for:
a. obtaining first spectral data from the individual's brain to determine the concentration of neurochemicals, includes the fucosylated glycans, in the brain;
b. subjecting the individual to therapy, which may include mere passage of time;
c. after step b., obtaining second spectral data from the individuals' brain to determine the concentration of neurochemicals includes the fucosylated glycans in the brain; and
d. comparing the second spectral data to the first spectral data to determine whether the concentration of fucosylated glycans in the second spectral data are changed from the concentration in the first spectral data and returning to the concentration of fucosylated glycans in the brain of a healthy person who is not experiencing pain.

9. A system for monitoring the response to treatment of an individual experiencing pain by detecting the level of chemical activity in the brain, comprising:
an MRS system for obtaining first spectral data from the individual's brain to determine the concentration of fucosylated glycans in the brain, and after subjecting the individual to therapy, which may include mere passage of time, obtaining second spectral data from the individuals' brain to determine the concentration of fucosylated glycans in the brain; and
a comparator for comparing the second spectral data to the first spectral data to determine whether the concentration of fucosylated glycans in the second spectral data are changed from the concentration in the first spectral data and returning to the concentration of fucosylated glycans in the brain of a healthy person who is not experiencing pain.

10. The system of claim 9, wherein the pain comprises at least one of acute pain, chronic pain, acute stress disorder, blast exposure and PTSD.

11. The system of claim 9, wherein the first and second spectral data are obtained using 2D COSY.

12. The system of claim 9, wherein the first and second spectral data determine the concentration of fucosylated glycans in a fully free form.

13. The system of claim 9, wherein the first and second spectral data determine the concentration of fucosylated glycans attached to another small molecule as a base pair.

14. The system of claim 9, including a non-volatile memory device for storing program steps for controlling at least one of obtaining spectral data, and comparing the spectral data.

15. The system of claim 9, wherein the comparator is located at a location remote from the MRS system.

16. The system of claim 15, wherein the comparator is located in the cloud.

17. A method for enabling monitoring the response of an individual experiencing pain to treatment, comprising:
Comparing the concentration of fucosylated glycans from an individual's brain from spectral data of the individual at second time period with the concentration of fucosylated glycans from an individual's brain from spectral data of the individual at an earlier first time period at which the individual was experiencing pain to determine whether any change in the concentration of fucosylated glycans occurred from the first time period to the second time period and whether the concentration of fucosylated glycans at the second time period is similar to that of a healthy person who is not experiencing pain.

18. The method of claim 17, wherein the pain comprises at least one of acute pain, chronic pain, acute stress disorder, blast exposure and post traumatic stress disorder (PTSD).

19. The method of claim 17, wherein the spectral data was obtained using 2D COrrelated Spectroscopy (COSY).

20. The method of claim 17, wherein the concentration of fucosylated glycans are in a fully free form.

21. The method of claim 17, wherein the fucosylated glycans are attached to another small molecule as a base pair.

22. The method of claim 17, where in the step of comparing is performed at a location remote from where the spectral data was obtained.

23. The method of claim 17, wherein the step of comparing is performed in the cloud.

24. A non-volatile memory for storing program steps for comparing the concentration of fucosylated glycans from an individual's brain from spectral data of the individual at second time period with the concentration of fucosylated glycans from an individual's brain from spectral data of the individual at an earlier first time period at which the individual was experiencing pain to determine whether any change in the concentration of fucosylated glycans occurred from the first time period to the second time period and whether the concentration of fucosylated glycans at the second time period is similar to that of a healthy person who is not experiencing pain.

25. A system for enabling monitoring the response of an individual experiencing pain to treatment, comprising a processor for comparing the concentration of fucosylated glycans from an individual's brain from spectral data of the individual at second time period with the concentration of fucosylated glycans from an individual's brain from spectral data of the individual at an earlier first time period at which the individual was experiencing pain to determine whether any change in the concentration of fucosylated glycans occurred from the first time period to the second time period and whether the concentration of fucosylated glycans at the second time period is similar to that of a healthy person who is not experiencing pain.

26. The system of claim 25, wherein the pain comprises at least one of acute pain, chronic pain, acute stress disorder, blast exposure and post traumatic stress disorder (PTSD)

27. The system of claim 25, wherein the spectral data was obtained using 2D Correlation Spectroscopy (COSY).

28. The system of claim 25, wherein the concentration of fucosylated glycans are in a fully free form.

29. The system of claim 25, wherein the fucosylated glycans are attached to another small molecule as a base pair.

30. The system of claim 25, wherein the processor is located at a location remote from the spectral data was obtained.

31. The system of claim 25, wherein the processor is in the cloud.
